(19) United States
(12) Patent Application Publication
(10) Pub. No.: US 2023/0197205 A1
(43) Pub. Date: Jun. 22, 2023

(54) BIORETROSYNTHETIC METHOD AND SYSTEM BASED ON AND-OR TREE AND SINGLE-STEP REACTION TEMPLATE PREDICTION

(71) Applicant: Wuhan University, Wuhan City (CN)

(72) Inventors: Juan LIU, Wuhan City (CN); Xiaolei ZHANG, Wuhan City (CN); Jing FENG, Wuhan City (CN)

(21) Appl. No.: 17/871,649

(22) Filed: Jul. 22, 2022

(30) Foreign Application Priority Data
Dec. 21, 2021 (CN) 202111570217.2

(51) Int. Cl. G16C 20/10; G16C 20/60; G16C 20/70

(52) U.S. Cl. CPC G16C 20/10; G16C 20/60; G16C 20/70

(57) ABSTRACT

The present disclosure provides a bioretrosynthetic method and system based on an AND-OR tree and single-step reaction template prediction. The method decomposes generation of a retrosynthetic pathway of a target molecule into multiple steps of the single-step reaction template prediction when conducting retrosynthesis on the target molecule. During the single-step reaction template prediction, a substrate molecule of the reaction template predicted in a previous step is used as a product molecule of a current reaction template to be predicted. Molecular characteristics of the product molecule are subjected to custom calculation using a SMILES sequence of the product molecule as an input, to be compatible with various single-step reaction template prediction models. The reaction template is determined according to a prediction result of the model, and extended based on a structure of the AND-OR tree. As a result, a potential synthetic pathway is found for the target molecule.

### CROSS REFERENCE TO RELATED APPLICATION

This patent application claims the benefit and priority of Chinese Patent Application No. 202111570217.2, filed on Dec. 21, 2021, the disclosure of which is incorporated by reference herein in its entirety as part of the present application.

### TECHNICAL FIELD

The present disclosure relates to the technical field of metabolic pathway analysis in biosynthesis, in particular to a bioretrosynthetic method and system based on an AND-OR tree and single-step reaction template prediction, belonging to use of the AND-OR tree and a machine learning method in the field of bioretrosynthesis.

### BACKGROUND ART

In the 1960s, Corey proposed the concept of retrosynthesis. A core idea of the retrosynthesis is to decompose a target molecule into simpler molecules that can produce the target molecule by a chemical reaction, and to repeat above process until all decomposed simpler molecules are commercially available. Up to now, chemical retrosynthesis has been very mature. Commercially available software has been developed to help synthesize high-value compounds. However, the software hardly take into account the principle of green chemistry, and the synthetic pathways they find may use or produce raw materials, catalysts, solvents, reagents, products, and by-products that are harmful to human health, community safety, and ecological environment. In the early 2000s, Hatzimanikatis V proposed the idea of bioretrosynthesis. A main difference from the chemical retrosynthesis is that the bioretrosynthesis restricts a process to metabolic reactions, and requires the final compounds to be the available precursors to the chassis strain. Designing putative metabolic pathways based on various chassis strains has also become an important topic in synthetic biology. Bioretrosynthesis involves designing, evaluating, and optimizing the metabolic pathways to produce high-value compounds from renewable resources and enzymes. Compared with the traditional chemical retrosynthesis, the bioretrosynthesis is more environmental-friendly and saves the cost of raw materials.

According to the statistics of MetaNetX database, among the 1,045,319 kinds of compound molecules and 75,699 reactions, there are 39,768 kinds of enzyme-catalyzed reactions, involving about 30,370 kinds of compound molecules, which accounting for only 2.9% of a total number of the compound molecules. There are still a large number of metabolic synthetic pathways of compound molecules to be explored. Traditional bioretrosynthesis-based pathway design methods require biologists to conduct a large number of wet experiments, by which a synthetic pathway is eventually found through a complete set of “design, build, test, and learn” processes. This work is extremely labor-intensive, material-cost, and time-cost. Therefore, it is of great practical significance to automate the bioretrosynthesis by means of computer technologies.

Existing bioretrosynthetic methods mainly construct biosynthetic pathways by searching for reactions or reaction templates whose products are similar to molecules to be predicted, which are not suitable for constructing biosynthetic pathways with long reaction steps due to the low computational efficiency. Moreover, most of the methods need complex parameter settings such as thermodynamics, cofactors, and enzyme performance, with a high domain knowledge requirement. In addition, the existing methods do not evaluate the correctness of metabolic reactions obtained by each step during the searching, and it is difficult to ensure an actual availability of the biosynthetic pathways eventually obtained.

### SUMMARY

The present disclosure proposes a bioretrosynthetic method and system based on an AND-OR tree and single-step reaction template prediction. Molecule nodes are selected through AND-OR tree searching, metabolic reaction templates that can generate product molecules are predicted based on a single-step reaction template prediction model, and the AND-OR tree can be extended, such that synthetic pathways can be generated. The present disclosure solves the technical problems of low efficiency and poor practical usability of the methods in the prior art.

The present disclosure provides a bioretrosynthetic method based on an AND-OR tree and single-step reaction template prediction, including the following steps:
S1: selecting an OR node from a pre-constructed AND-OR tree, and using a molecule corresponding to the OR node as a product molecule to be predicted; where the pre-constructed AND-OR tree includes two types of nodes, an AND node and the OR node; the AND node represents a reaction template, and the OR node represents a molecule;
S2: predicting k templates in a preset template set that are most likely to synthesize the product molecule using a pre-constructed single-step reaction template prediction model, forming a template set Top-k, and assigning a weight value ranged between 0 and 1 to each template; where the preset template set is constructed based on a metabolic reaction structure in a known metabolic reaction data set;
S3: expanding the pre-constructed AND-OR tree, specifically including: adding each template in the Top-k as a new AND node to the AND-OR tree to obtain newly-added k AND nodes, and using the OR node selected in step S1 as a parent node of the newly-added k AND nodes; adding each reaction substrate molecule corresponding to each newly-added AND node to the AND-OR tree as an OR node to obtain newly-added OR nodes, with the newly-added AND node as a parent node of the newly-added OR nodes; and
S4: determining whether there is an AND node in step S3, where a substrate molecule corresponding to a child node of the AND node belongs to a known metabolite set: Sink-Compounds set; if there is an AND node, finding a biosynthetic pathway, stopping an iterative retrosynthetic process, and generating the biosynthetic pathway; if there is no AND node, determining whether the maximum number of iterations has been reached; if the maximum number of iterations is reached, stopping the iterative retrosynthetic process; if the maximum number of iterations is not reached, repeating steps S1 to S4 until a biosynthetic pathway is found or the maximum number of iterations is reached.

Preferably, in step S1: in the AND-OR tree, a root node and a leaf node each may be an OR node, and an intermediate node may be an AND node or an OR node; a child node of each AND node may be an OR node, representing all substrate molecules in a reaction template; a child node of each non-leaf OR node in the AND-OR tree may be an AND node; each AND node may represent a reaction template capable of producing a molecule corresponding to a parent node of the AND node, and the root node of the AND-OR tree may be a target molecule node for quasi-prediction of a biosynthetic pathway; and an initially-constructed AND-OR tree may include only one root node, corresponding to the target molecule of a biosynthetic pathway to be predicted.

Preferably, in step S1, each node of the AND-OR tree may have a weight value; a weight value of the AND node may be a weight value of a corresponding reaction template, indicating a prediction probability of the corresponding reaction template; and OR nodes other than the root node each may have a weight value of the parent node of the OR nodes, and the root node may have a weight value of 1.

Preferably, in step S1, the selected OR node may be a leaf node in the AND-OR tree that does not belong to the known Sink-Compounds set and has a maximum weight value; and if there are a plurality of the leaf nodes with the maximum weight value, one of the leaf nodes may be selected randomly.

Preferably, the pre-constructed single-step reaction template prediction model may be a multi-classification model based on machine learning; and step S2 may specifically include:
S2.1: predicting a probability that all reaction templates in the preset template set are capable of producing an input product molecule using the constructed multi-classification model; and
S2.2: selecting top k reaction templates with the highest probabilities to form the template set Top-k, and setting a weight value of each reaction template in the template set Top-k as a corresponding probability value.

Preferably, in step S3, each reaction substrate molecule of the newly-added AND nodes may be obtained by calling a function in an open source library RDChiral; and in the function, an input parameter may be a SMILES sequence of the reaction template and the product molecule, and an output may be a list of the corresponding substrate molecules.

Preferably, in step S4, a process of generating the biosynthetic pathway may include the following steps:
(1) checking whether each leaf node in the AND-OR tree is in the Sink-Compounds set; marking the leaf node in the Sink-Compounds as “true”, and marking the leaf node not in the Sink-Compounds as “false”;
(2) for a non-leaf AND node in the AND-OR tree, marking the non-leaf AND node as “true” if and only if each child node of the non-leaf AND node is marked as “true”, otherwise marking the non-leaf AND node as “false”; and for a non-leaf OR node in the AND-OR tree, marking the non-leaf OR node as “true” if and only if the non-leaf OR node includes at least one child node marked as “true”, otherwise marking the non-leaf OR node as “false”; and
(3) if the root node is marked as “false”, it indicates that no synthetic pathway has been found, outputting “No Solution”; otherwise, deleting all nodes marked as “false” in the AND-OR tree, and remaining a subtree representing a synthetic pathway of a target molecule.

The present disclosure further provides a bioretrosynthetic system based on an AND-OR tree and single-step reaction template prediction, including:
a retrosynthesis planning module used for selecting an OR node from a pre-constructed AND-OR tree, and using a molecule corresponding to the OR node as a product molecule to be predicted; where the pre-constructed AND-OR tree includes two types of nodes, an AND node and the OR node; the AND node represents a reaction template, and the OR node represents a molecule;
a reaction template prediction module used for predicting k templates in a preset template set that are most likely to synthesize the product molecule using a pre-constructed single-step reaction template prediction model, forming a template set Top-k, and assigning a weight value ranged between 0 and 1 to each template; where the preset template set is constructed based on a metabolic reaction structure in a known metabolic reaction data set;
an AND-OR tree extension module used for expanding the pre-constructed AND-OR tree, specifically including: adding each template in the Top-k as a new AND node to the AND-OR tree to obtain newly-added k AND nodes, and using the OR node selected in the retrosynthesis planning module as a parent node of the newly-added k AND nodes; adding each reaction substrate molecule corresponding to each newly-added AND node to the AND-OR tree as an OR node to obtain newly-added OR nodes, with the newly-added AND node as a parent node of the newly-added OR nodes; and
a biosynthetic pathway generation module used for determining whether there is an AND node in the AND-OR tree obtained by the AND-OR tree extension module, where a substrate molecule corresponding to a child node of the AND node belongs to a known metabolite set: Sink-Compounds set; if there is an AND node, finding a biosynthetic pathway, stopping an iterative retrosynthetic process, and generating the biosynthetic pathway; if there is no AND node, determining whether the maximum number of iterations has been reached; if the maximum number of iterations is reached, stopping the iterative retrosynthetic process; if the maximum number of iterations is not reached, repeating steps of the retrosynthesis planning module to the biosynthetic pathway generation module until a biosynthetic pathway is found or the maximum number of iterations is reached.

Preferably, the system may further include: a biosynthetic pathway visualization module for visually displaying an obtained biosynthetic pathway of a target molecule.

The foregoing one or more technical solutions in the embodiments of this application have at least one or more of the following technical effects:

The present disclosure provides a bioretrosynthetic method based on an AND-OR tree and single-step reaction template prediction. An OR node is selected as the product molecule to be predicted; the product molecule to be predicted is predicted using a pre-constructed single-step reaction template prediction model, to obtain the k templates that are most likely to synthesize the product molecule in the preset template set; the AND-OR tree is expanded; the prediction of the next reaction template is planned based on and the AND-OR tree search; and the biosynthetic pathway of the target molecule is finally generated. In the present disclosure, the single-step reaction template prediction model is adopted, without complicated parameter settings such as thermodynamics, cofactors, and enzyme performance or complex domain knowledge, to predict reaction templates, which can improve the efficiency. Meanwhile, planning of the template predictions based on the AND-OR tree search can help to select the metabolic reactions most likely to participate in the biosynthetic pathway, thus improving the actual availability of the generated biosynthetic pathway.

In addition, the present disclosure further proposes a bioretrosynthetic system based on an AND-OR tree and single-step reaction template prediction, which can assist biologists to find a potential metabolic pathway quickly, thereby reducing experimental costs and improving experimental efficiency.

### BRIEF DESCRIPTION OF THE DRAWINGS

To describe the technical solutions in examples of the present disclosure or in the prior art more clearly, the accompanying drawings required for describing the examples or the prior art will be briefly described below. Apparently, the accompanying drawings in the following description show some examples of the present disclosure, and a person of ordinary skill in the art may still derive other drawings from these accompanying drawings without creative efforts.

FIG. 1 shows a flow chart of a bioretrosynthetic method based on an AND-OR tree and single-step reaction template prediction in examples of the present disclosure;

FIG. 2 shows an operation chart of constructing and expanding the AND-OR tree in the method in examples of the present disclosure; where a left side shows a node s in the AND-OR tree as a product molecule, and nodes with a maximum weight include a, b, and c; a right side shows an AND-OR tree expanded by randomly selecting the node a; circles represent OR nodes, rectangles represent AND nodes, pentagons represent quasi-expansion nodes, and numbers on a right side of each node represent a node value;

FIG. 3 shows a SMILES sequence of product/substrate molecules and a molecular diagram thereof in examples of the present disclosure;

FIG. 4 shows a schematic diagram of a reaction template obtained by single-step reaction template prediction and a corresponding reaction thereof in examples of the present disclosure; where an upper part shows the reaction template, and a lower part shows the reaction corresponding to the reaction template;

FIG. 5 shows a schematic diagram of a biosynthetic pathway of a target molecule obtained by a method in examples of the present disclosure; where a circle represents the target product molecule, rectangles represent intermediate molecules, and diamonds represent Sink-Compounds; and

FIG. 6 shows a schematic diagram of modules of a bioretrosynthetic system based on an AND-OR tree and single-step reaction template prediction in examples of the present disclosure.

### DETAILED DESCRIPTION OF THE EMBODIMENTS

Aiming at deficiencies of the prior art, the present disclosure provides a bioretrosynthetic method based on an AND-OR tree and single-step reaction template prediction. A single-step reaction template prediction model is used to predict metabolic reaction templates that can produce product molecules and expand the AND-OR tree, and plan a next reaction template based on AND-OR tree searching to predict and generate a final biosynthetic pathway. The method adopts a single-step reaction template prediction model, without complex domain knowledge, to predict metabolic reaction templates in sequence, and can predict the Top-k reaction templates of product molecules. Meanwhile, planning template predictions by the AND-OR tree helps to select metabolic reactions most likely to participate in the biosynthetic pathway, thus improving the actual availability of the target biosynthetic pathway.

The present disclosure further proposes a bioretrosynthetic system based on an AND-OR tree and single-step reaction template prediction, which can assist biologists to find a potential metabolic pathway quickly, thereby reducing experimental costs and improving experimental efficiency.

In order to make the objectives, technical solutions and advantages of the embodiments of the present disclosure clearer, the technical solutions in the embodiments of the present disclosure will be clearly and completely described below in conjunction with the drawings in the embodiments of the present disclosure. Obviously, the described embodiments are some, rather than all of the embodiments of the present disclosure. All other embodiments obtained by those of ordinary skill in the art based on the embodiments of the present disclosure without creative efforts should fall within the protection scope of the present disclosure.

### Example 1

The present disclosure provides a bioretrosynthetic method based on an AND-OR tree and single-step reaction template prediction, including the following steps:
S1: selecting an OR node from a pre-constructed AND-OR tree, and using a molecule corresponding to the OR node as a product molecule to be predicted; where the pre-constructed AND-OR tree includes two types of nodes, an AND node and the OR node; the AND node represents a reaction template, and the OR node represents a molecule;
S2: predicting k templates in a preset template set that are most likely to synthesize the product molecule using a pre-constructed single-step reaction template prediction model, forming a template set Top-k, and assigning a weight value ranged between 0 and 1 to each template; where the preset template set is constructed based on a metabolic reaction structure in a known metabolic reaction data set;
S3: expanding the pre-constructed AND-OR tree, specifically including: adding each template in the Top-k as a new AND node to the AND-OR tree to obtain newly-added k AND nodes, and using the OR node selected in step S1 as a parent node of the newly-added k AND nodes; adding each reaction substrate molecule corresponding to each newly-added AND node to the AND-OR tree as an OR node to obtain newly-added OR nodes, with the newly-added AND node as a parent node of the newly-added OR nodes; and
S4: determining whether there is an AND node in step S3, where a substrate molecule corresponding to a child node of the AND node belongs to a known metabolite set: Sink-Compounds set; if there is an AND node, finding a biosynthetic pathway, stopping an iterative retrosynthetic process, and generating the biosynthetic pathway; if there is no AND node, determining whether the maximum number of iterations has been reached; if the maximum number of iterations is reached, stopping the iterative retrosynthetic process; if the maximum number of iterations is not reached, repeating steps S1 to S4 until a biosynthetic pathway is found or the maximum number of iterations is reached.

Specifically, step S1 is retrosynthetic planning, S2 is reaction template prediction, S3 is AND-OR tree expansion, and S4 is biosynthetic pathway generation.

The prediction of a preset template set R is to predict a set required for the bioretrosynthetic pathway; each reaction template in the set R mainly includes one or more substrate molecules and one product molecule, meaning that one or more of the substrate molecules can produce the corresponding product molecule after reaction.

In S1, an initial AND-OR tree includes only one root node, which is an OR node representing a target molecule to be predicted, and the root node is expanded.

In the present disclosure, generation of the retrosynthetic pathway of the target molecule is decomposed into multiple steps of single-step reaction template prediction. In the single-step reaction prediction, a first input product molecule is the target molecule, and a subsequent product molecule input to the model is an intermediate molecule in the reaction chain; that is, a substrate molecule of a reaction template predicted in a previous step is used as a product molecule of the current reaction template to be predicted. For example, if a synthetic pathway of a molecule C needs to be predicted, the molecule C is input as a target molecule into the single-step reaction template prediction model, and k templates that are most likely to synthesize the molecule C are obtained in the preset template set; taking a template R1 corresponding to the highest probabilities as an example, a reaction corresponding to the reaction template is selected; for example, substrates of the reaction are A and B, and the A and B can become C through the reaction template R1; the next single-step reaction prediction is conducted, the substrate A is input as a product molecule into the single-step reaction template prediction model, and k templates that can synthesize A are predicted, and the processing process for B is similar to that of B; in a similar fashion, until conditions of step S3 are met: a biosynthetic pathway that can generate the molecule C is found, or the maximum number of iterations is reached.

Preferably, in step S1: in the AND-OR tree, a root node and a leaf node each may be an OR node, and an intermediate node may be an AND node or an OR node; a child node of each AND node may be an OR node, representing all substrate molecules in a reaction template; a child node of each non-leaf OR node in the AND-OR tree may be an AND node; each AND node may represent a reaction template capable of producing a molecule corresponding to a parent node of the AND node, and the root node of the AND-OR tree may be a product molecule node for quasi-prediction of a biosynthetic pathway.

Specifically, the AND-OR tree includes two types of nodes, where the AND node is a node representing the reaction template, also known as a template node, and the OR node represents a molecule, also known as a molecular node.

Preferably, in step S1, each node of the AND-OR tree may have a weight value; a weight value of the AND node may be a weight value of a corresponding reaction template, indicating a prediction probability of the corresponding reaction template; and OR nodes other than the root node each may have a weight value of the parent node of the OR nodes, and the root node may have a weight value of 1.

Preferably, in step S1, the selected OR node may be a leaf node in the AND-OR tree that does not belong to the known Sink-Compounds set and has a maximum weight value; and if there are a plurality of the leaf nodes with the maximum weight value, one of the leaf nodes may be selected randomly.

Preferably, the pre-constructed single-step reaction template prediction model may be a multi-classification model based on machine learning; and step S2 may specifically include:
S2.1: predicting a probability that all reaction templates in the preset template set are capable of producing an input product molecule using the constructed multi-classification model; and
S2.2: selecting top k reaction templates with the highest probabilities to form the template set Top-k, and setting a weight value of each reaction template in the template set Top-k as a corresponding probability value.

Preferably, in step S3, each reaction substrate molecule of the newly-added AND nodes may be obtained by calling a function in an open source library RDChiral; and in the function, an input parameter may be a SMILES sequence of the reaction template and the product molecule, and an output may be a list of the corresponding substrate molecules.

The function in RDChiral is an rdchiralRunText function.

Preferably, in step S4, a process of generating the biosynthetic pathway may include the following steps:
1) checking whether each leaf node in the AND-OR tree is in the Sink-Compounds set; marking the leaf node in the Sink-Compounds as “true”, and marking the leaf node not in the Sink-Compounds as “false”;
2) for a non-leaf AND node in the AND-OR tree, marking the non-leaf AND node as “true” if and only if each child node of the non-leaf AND node is marked as “true”, otherwise marking the non-leaf AND node as “false”; and for a non-leaf OR node in the AND-OR tree, marking the non-leaf OR node as “true” if and only if the non-leaf OR node includes at least one child node marked as “true”, otherwise marking the non-leaf OR node as “false”; and
3) if the root node is marked as “false”, it indicates that no synthetic pathway has been found, outputting “No Solution”; otherwise, deleting all nodes marked as “false” in the AND-OR tree, and remaining a subtree representing a synthetic pathway of a target molecule.

In the specific implementation, the Sink-Compounds refers to precursors in a medium or metabolites available in a host organism/strain, the data of which are derived from a BiGG database; by integrating data of iML1515, iJO1366, E. coli core metabolism and Bacillus subtilis iYO844, a Sink-Compounds data set is constructed to determine whether the substrate molecules for bioretrosynthesis are available and whether further iterative retrosynthesis is required.

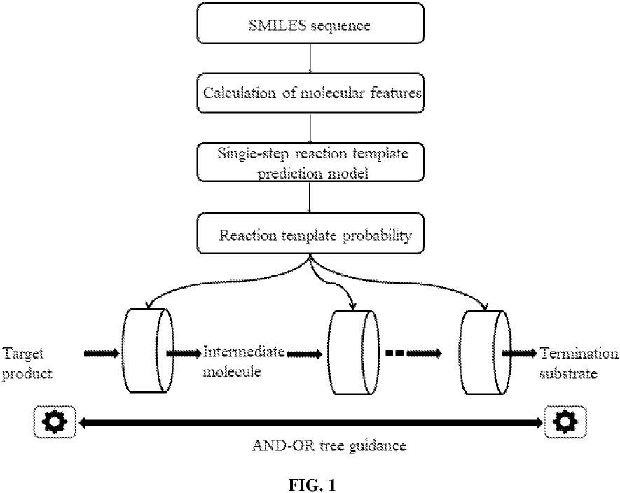

FIG. 1 shows a flow chart of a bioretrosynthetic method based on an AND-OR tree and single-step reaction template prediction in examples of the present disclosure.

In specific use, the AND-OR tree constructed in S1 includes two types of nodes: AND and OR nodes. The AND node represents a metabolic reaction template node, and the OR node represents a molecular node. In the AND-OR tree, the root node and the leaf node each are OR nodes, and the intermediate nodes have the AND nodes and OR nodes. In the AND-OR tree, each child node of each AND node is an OR node, representing all substrate molecules in a reaction template. For each non-leaf OR node in the AND-OR tree, child nodes are the AND nodes, and each AND node represents a reaction template that can generate a molecule corresponding to its parent node. The root node of the AND-OR tree is a product molecule node for quasi-prediction of a biosynthetic pathway. In the AND-OR tree, each node has a weight value. A weight value of the AND node is a weight value of a corresponding reaction template; and OR nodes other than the root node each have a weight value of the parent node except for the OR nodes. The root node has a weight value of 1.

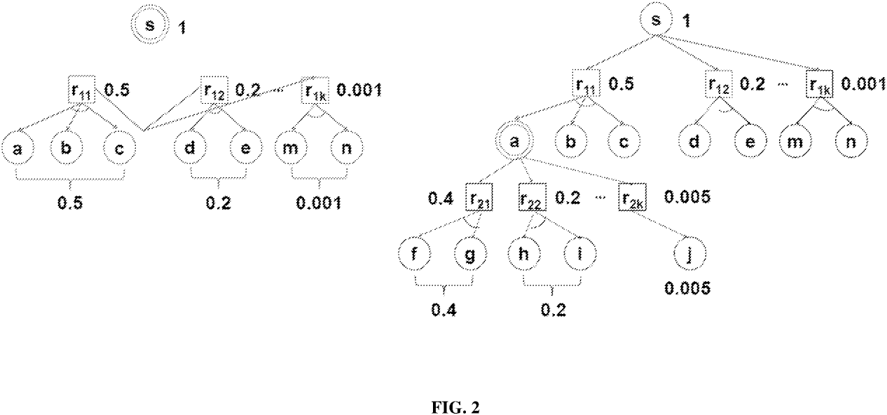

In step S1, the OR node selected in the AND-OR tree is a leaf node that does not belong to the Sink-Compounds set and has the maximum weight value. If there are more than one OR node, one of the OR nodes is randomly selected. Referring to FIG. 2, the left part is the current AND-OR tree. An OR node a that has the maximum weight and is not in the Sink-Compounds set is selected as a new molecule node of the product to be predicted, realizing planning the single-step reaction template prediction for a next step.

In step S2, the single-step reaction template prediction model B includes two steps: (1) simultaneously predicting a probability that all reaction templates in a known metabolic reaction template set R can produce the input product molecules using a multi-classification model constructed based on machine learning; and (2) selecting the top k templates with the highest probabilities to form a set Top-k, and setting a weight value of each template as a probability value.

In the specific implementation, the metabolic reaction template set R in (1) is constructed based on a MetaNetX metabolic reaction database. The database is cleaned; and a template_extractor function in the open source library RDChiral is called to obtain a reaction template corresponding to the reaction, where in the function, an input is a specific metabolic reaction, and an output is the corresponding reaction template. Data cleaning and template set generation specifically includes:
1) if the reaction has multiple products, the reaction is represented as multiple reactions, and each reaction has only one product;
2) the reaction represented by the corresponding SMILES sequence cannot be found by removing the product and the substrate;
3) the reaction with a product being groups and cluster molecules is removed;
4) the reaction with a product being cofactors (such as ions, water, carbon dioxide, ATP, and NADP) is removed;
5) the reaction with more than 3 substrates is removed;
6) the reaction with products or substrates having a SMILES sequence of longer than 250 is removed;
7) atomic mapping information is added to all the reactions after cleaning using an RXNMapper tool, and reactions that failed to map are removed;
8) a reaction template for each reaction is extracted using the template_extractor function in the RDChiral, and reactions that fail to extract the templates are removed; and
9) for each reaction template, an rdchiralRunText function in RDChiral is called to obtain all possible substrates, and reaction templates that cannot obtain a real substrate in the reaction corresponding to the template are removed.

In specific implementation, the single-step reaction template prediction model B can be constructed by a traditional machine learning algorithm or by a deep learning algorithm.

As a specific embodiment, the single-step reaction template prediction model B is constructed by using the traditional machine learning algorithm and the deep learning algorithm separately. Specifically, the traditional machine learning algorithm adopts Gaussian Naive Bayes classifier, and the deep learning algorithm adopts the convolutional neural network ResNet50; construction of the single-step reaction template prediction model B based on two algorithms will be described as an example. The model input is a product molecule to be predicted represented by the SMILES sequence, and the model output is a probability that each reaction template in the metabolic reaction template set R can generate the product molecule.

### Specific Example 1

A training process of the single-step reaction template prediction model B using the traditional machine learning algorithm specifically included the following steps:

(1) Obtain training samples: the product molecules corresponding to all reactions in the reaction template set were collected, and the reaction templates corresponding to product molecules were labeled as their categories after deduplication.

(2) Determination of feature attributes: the molecular features of the product molecule were calculated, the molecular features were divided into multiple discrete feature attributes x, and then input to a Gaussian Naive Bayes classifier.

(3) The classifier calculated a frequency of occurrence of each category in the training sample, and the frequency was used as a probability P(yi) of each category, where y represented the category, yi represented the ith category, and P(yi) represented the probability that the training sample belonged to the ith category.

(4) The classifier calculated a conditional probability P(xlyi) of all divisions of each feature attribute x in the training sample.

(5) The classifier calculated P(xlyi)·P(yi) for each category, which was a probability that each reaction template could synthesize product molecules.

In step (2), the molecular feature of the product molecule specifically referred to a MACCSKeys fingerprint, with a total of 166 features; and each feature had a corresponding specific meaning, for example, the first feature indicated whether there were isomers, the 14th feature indicated whether there was a disulfide bond. The MACCSKeys fingerprint with a length of 167 was calculated by an open source toolkit RDKit, where a 0th bit was a placeholder and did not provide any information, such that the 0th bit was deleted and only 166 features were retained. Correspondingly, each of the 166 features was taken as a feature attribute x,x□ {0,1}.

In step (4), the conditional probability P(xlyi) of the feature attribute x was calculated by assuming that the feature attribute x obeyed the Gaussian distribution, and was calculated according to a probability density function of the Gaussian distribution. The function was specifically as follows:

$$\text{P} \left(\text{x}\right) = \frac{1}{σ \sqrt{2 π}} {\text{e}}^{\frac{{\left(\text{x} - μ\right)}^{2}}{{20}^{2}}}$$

x represented a feature attribute value, σ represented a standard deviation of the Gaussian distribution, and µ represents a mean of the Gaussian distribution.

### Specific Example 2

A training process of the single-step reaction template prediction model B using the deep learning algorithm specifically included the following steps:

(1) Obtain training samples: the product molecules corresponding to all reactions in the reaction template set were collected, and the reaction templates corresponding to product molecules were labeled as their categories after deduplication.

(2) The molecular features of the product molecule were calculated and input into a convolutional neural network ResNet50 classifier.

(3) The classifier output a probability that each reaction template could synthesize product molecules.

In step (2), the molecular feature F₂ of the product molecule was constructed based on the enhanced expansion of the molecular feature F₁ in the specific example 1. Specifically, the molecular feature F₁ at position 166 was reversed to obtain a new molecular feature Fi′; the molecular features F1 and F1′ were longitudinally spliced to obtain an enhanced molecular feature F_{aug} with dimension of [2,166]; the molecular feature F_{aug} was repeated longitudinally to obtain a final molecular feature F with dimension of [166,166].

In step (2), to adapt to the dimension of the molecular feature F, the number of input channels of the ResNet50 classifier was changed to 1, and AvgPool2d was changed to AdaptiveAvgPool2d. Furthermore, the output of the last fully-connected layer was modified to the number of reaction template categories.

Specifically, a main idea of ResNet50 was residual learning, which proposed two mapping methods, namely residual mapping and self mapping. An objective function combined these two parts, and a specific formula was as follows:

$$\text{h} \left(\text{x}\right) = \left(\text{h} \left(\text{x}\right) \left(\text{-x}\right)\right) + \text{x}$$

h(x) represented the objective function, x represented self mapping, and h(x)-x represented the residual mapping.

ResNet50 used a ReLU activation function, which was specifically expressed as:

$$\text{f} \left(\text{x}\right) = max \left(0 , \text{x}\right)$$

x represented an input of the neuron, which could turn all negative values into 0, while positive values remained unchanged; this unilateral inhibitory function enabled sparse activation of neurons in neural networks.

In the specific implementation, the data set of single-step reaction template prediction models in the specific example 1 and the specific example 2 included a given product molecule, a corresponding generated substrate molecule, and a reaction formed by the two. The data was derived from a MetaNetX database. The database stored metabolites and associated biochemical reactions, provided cross-links between major public biochemical and genome-scale metabolic network databases, and contained approximately 80,000 single-step reactions (including reactions without chemical structures). Therefore, the data was subjected to multi-layer cleaning; the data size was 30,986 after cleaning, involving 25,415 reactions, where 21,704 reactions had enzyme catalysis information, and a total of 15,930 reaction templates were extracted.

In the implementation of specific example 2, the training process of the ResNet50 classifier was to find an optimal value of the model according to a loss function and a gradient descent method. The loss function adopted a cross-entropy loss function, which was used to quantify a difference between model prediction and true labels; and the gradient descent method used an adaptive momentum estimation algorithm Adam to find a set of parameters that minimize the structural risk.

The cross-entropy loss function was specifically expressed as:

$$\text{J =} - \left[\text{ylog} \hat{\text{y}} \text{+} \left(1 - \text{y}\right) log \left(1 - \hat{\text{y}}\right)\right]$$

y represented the true labels, with a value of 0 or 1, and ^{ŷ} represented a probability that the sample was predicted to be positive; a greater difference between a predicted output and y led to a greater value of J.

The gradient descent method adopted the adaptive momentum estimation algorithm Adam, which could iteratively update neural network weights based on the training data. The Adam algorithm can be considered as a combination of RMSprop and Momentum, which not only uses a momentum method to update parameters, but also adaptively adjusts a learning rate. In back-propagation of the neural network, the momentum update method no longer only relies on a descending gradient of current parameters to update the parameters, but also relies on the previous epochs of parameters and the descending gradient of the parameters to update the current parameters. Therefore, when an objective function is obtained, the descending speed can be slowed down, such that it is easier to find the optimal value, and the oscillation in the gradient descent process that seriously affects the optimization speed can be effectively alleviated. At the t-th iteration, a parameter update formula is as follows:

$${\text{θ}}_{\text{t+1}} = {\text{θ}}_{\text{t}} \text{-} \text{η} ⋅ Δ \text{J} \left({\text{θ}}_{\text{t}}\right)$$

η was a learning rate, θt was a parameter of the t-th round, J(θₜ) was a loss function, and ΔJ(θₜ) was an updated gradient; an actual update difference of each parameter depended on a weighted average of the gradients in the most recent period, θₜ₊₁ represented the parameters of the t+1th round.

In the implementation of specific example 2, when training the single-step reaction template prediction model, the training set, validation set, and test set were based on the data set of the single-step reaction template prediction model, and randomly divided according to a ratio of 8:1:1, and a training round number L was set to 50; gradient descent calculation was conducted using the Adam optimizer, at an initial learning rate of 0.001 and a batch_size value of 1024; and parameters and results of the round with the smallest loss value in the validation set were obtained after L epochs of training.

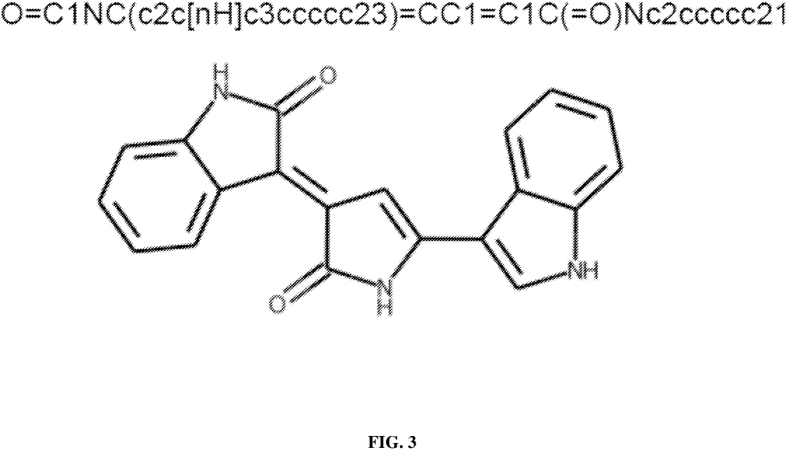

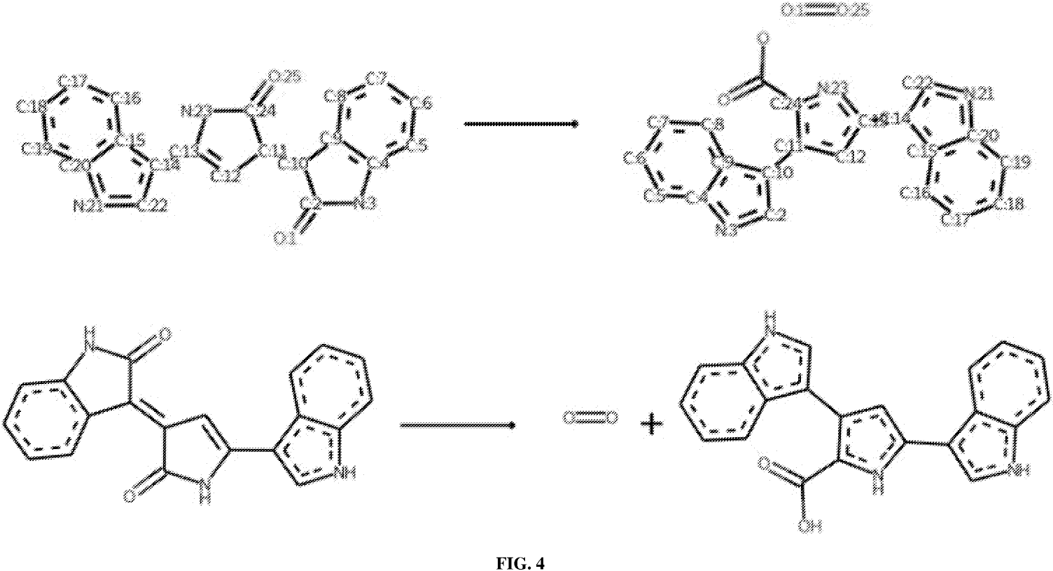

Referring to FIG. 3 to FIG. 4, FIG. 3 shows a SMILES sequence of product/substrate molecules and a molecular diagram thereof in specific examples 1 and 2; and FIG. 4 shows a schematic diagram of a reaction template obtained by single-step reaction template prediction and a corresponding reaction thereof in specific examples 1 and 2.

In an implementation, a k value of the Top-k in step S2 was set to 50, and the maximum number of iterations in step S4 was set to 50.

In step S3, each reaction substrate of an AND node ri was obtained by calling a rdchiralRunText function in an open source library RDChiral; and in the function, an input parameter was a SMILES sequence of the reaction template and the product molecule, and an output was a list of the corresponding substrate molecules.

Referring to the right side in FIG. 2, a leaf OR node “a” was selected in the left side, a relevant template was obtained by the prediction model in step S2, and an AND-OR tree was obtained after expanding the template in step S3.

In the example, the Sink-Compounds referred to precursors in the culture medium or metabolites available in a host organism/strain, the data of which were derived from the BiGG database; the Sink-Compounds set was constructed by integrating data of iML1515, iJO1366, E. coli core metabolism and Bacillus subtilis iYO844, and used to determine whether the substrate molecules for bioretrosynthesis were available and whether further iterative process of retrosynthesis was required. The specific data distribution was shown in Table 1.

TABLE 1

Number of Sink Compounds of various strains

| iML1515 | iJ01366 | iY0844 | E. coli core metabolism |
|---|---|---|---|
| 738 | 727 | 466 | 53 |

The biosynthetic pathway of step S4 was obtained by searching a finally obtained AND-OR tree. A process included the following steps:
1) checking whether each leaf node in the AND-OR tree is in the Sink-Compounds set; marking the leaf node in the Sink-Compounds as “true”, and marking the leaf node not in the Sink-Compounds as “false”;
2) for a non-leaf AND node in the AND-OR tree, marking the non-leaf AND node as “true” if and only if each child node of the non-leaf AND node is marked as “true”, otherwise marking the non-leaf AND node as “false”; and for a non-leaf OR node in the AND-OR tree, marking the non-leaf OR node as “true” if and only if the non-leaf OR node includes at least one child node marked as “true”, otherwise marking the non-leaf OR node as “false”; and
3) if the root node is marked as “false”, it indicates that no synthetic pathway has been found, outputting “No Solution”; otherwise, deleting all nodes marked as “false” in the AND-OR tree, and remaining a subtree representing a synthetic pathway of a target molecule.

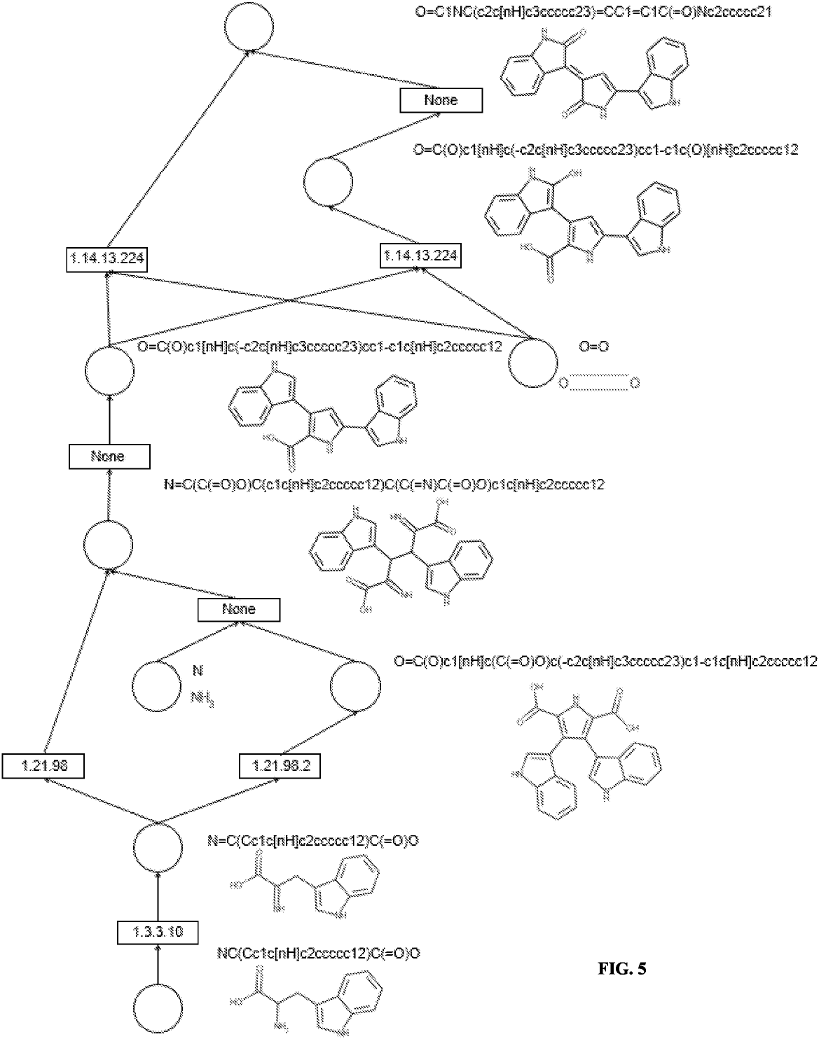

FIG. 5 shows a schematic diagram of a biosynthetic pathway of a target molecule obtained by a method.

Compared with the prior art, the present disclosure has the following beneficial effects:

1. The method is a first method in the field of bioretrosynthesis that combines AND-OR tree and single-step reaction template prediction model for bioretrosynthesis prediction, without complex domain parameter settings. The method solves the problems of high experimental cost and time-consuming of traditional pathway design methods, and can assist biologists to find potential synthetic pathways more quickly.

2. The method adopts an AND-OR tree structure to represent the retrosynthetic planning process, and the reactions can appear as nodes in the tree explicitly, which can capture a relationship between candidate reactions and substrate molecules. The method addresses a sparsity of variance estimates brought about by representing each node as a set of molecules in a Monte Carlo tree.

### Example 2

The present disclosure provides a bioretrosynthetic system based on an AND-OR tree and single-step reaction template prediction, including:
a retrosynthesis planning module used for selecting an OR node from a pre-constructed AND-OR tree, and using a molecule corresponding to the OR node as a product molecule to be predicted; where the pre-constructed AND-OR tree includes two types of nodes, an AND node and the OR node; the AND node represents a reaction template, and the OR node represents a molecule;
a reaction template prediction module used for predicting k templates in a preset template set that are most likely to synthesize the product molecule using a pre-constructed single-step reaction template prediction model, forming a template set Top-k, and assigning a weight value ranged between 0 and 1 to each template; where the preset template set is constructed based on a metabolic reaction structure in a known metabolic reaction data set;
an AND-OR tree extension module used for expanding the pre-constructed AND-OR tree, specifically including: adding each template in the Top-k as a new AND node to the AND-OR tree to obtain newly-added k AND nodes, and using the OR node selected in the retrosynthesis planning module as a parent node of the newly-added k AND nodes; adding each reaction substrate molecule corresponding to each newly-added AND node to the AND-OR tree as an OR node to obtain newly-added OR nodes, with the newly-added AND node as a parent node of the newly-added OR nodes; and
a biosynthetic pathway generation module used for determining whether there is an AND node in the AND-OR tree obtained by the AND-OR tree extension module, where a substrate molecule corresponding to a child node of the AND node belongs to a known metabolite set: Sink-Compounds set; if there is an AND node, finding a biosynthetic pathway, stopping an iterative retrosynthetic process, and generating the biosynthetic pathway; if there is no AND node, determining whether the maximum number of iterations has been reached; if the maximum number of iterations is reached, stopping the iterative retrosynthetic process; if the maximum number of iterations is not reached, repeating steps of the retrosynthesis planning module to the biosynthetic pathway generation module until a biosynthetic pathway is found or the maximum number of iterations is reached.

Specifically, before the synthetic pathway generation, there are some pre-operations, reaction template set construction, Sink-Compounds set construction and data preprocessing. Specifically, the system includes: a reaction template set construction module for constructing a template set required for biosynthetic pathway prediction, a Sink-Compounds set construction module for constructing available precursors in media or metabolite sets available in host organisms/strains, and a data preprocessing module for generating molecular features (such as MACCSKeys fingerprints) based on the SMILES sequence of the molecule before the product molecule is input into the model.

In summary, the present disclosure provides a bioretrosynthetic method based on an AND-OR tree and single-step reaction template prediction. The method decomposes generation of a retrosynthetic pathway of a target molecule into multiple steps of the single-step reaction template prediction when conducting retrosynthesis on the target molecule. During the single-step reaction template prediction, a substrate molecule of the reaction template predicted in a previous step is used as a product molecule of a current reaction template to be predicted. Molecular characteristics of the product molecule are subjected to custom calculation using a SMILES sequence of the product molecule as an input, to be compatible with various single-step reaction template prediction models. According to a probability that each reaction template predicted by the model can synthesize product molecules, the top k reaction templates with the highest probabilities are selected, and then extended based on the AND-OR tree structure. As a result, a potential synthetic pathway is found for the target molecule. The present disclosure further provides a system based on the bioretrosynthetic method based on an AND-OR tree and single-step reaction template prediction. The system realizes the automation of retrosynthesis of metabolic pathways for the target molecules through modular processing procedures, such as reaction template set construction, Sink-Compounds set construction, data preprocessing, reaction template prediction, AND-OR tree expansion, retrosynthetic planning, biosynthetic pathway generation, and visualization. In the present disclosure, a speed of finding a feasible synthetic pathway is significantly improved, without manually-set complex parameters, which can assist biologists to find a potential metabolic pathway more quickly, thereby reducing experimental costs and improving experimental efficiency.

In an implementation, the system further includes: a biosynthetic pathway visualization module for visually displaying an obtained biosynthetic pathway.

The retrosynthesis planning module is used to: search the leaf nodes of the current AND-OR tree, select a leaf OR node as a product molecule of the next round of single-step reaction template prediction, thus planning and searching of the retrosynthetic pathway; the reaction template prediction module is used to: predict the k templates that are most likely to produce product molecules by a constructed prediction model; and the AND-OR tree expansion module is used to: according to a type of the node to be expanded, expand the AND-OR tree based on an OR node expansion template or an AND node expansion template separately. The biosynthetic pathway generation module is used to: search the final AND-OR tree to obtain the biosynthetic pathway of the target molecule; and the biosynthetic pathway visualization module is used to: visualize the final biosynthetic pathway for reference by experts in the field of biosynthesis.

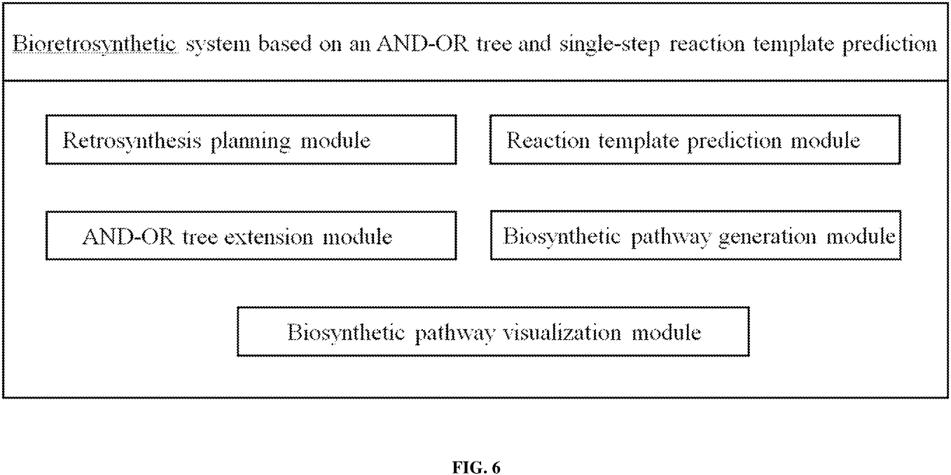

FIG. 6 shows a schematic diagram of modules of a bioretrosynthetic system based on an AND-OR tree and single-step reaction template prediction in examples of the present disclosure.

The system introduced in Example 2 of the present disclosure is a system used to implement the bioretrosynthetic method based on an AND-OR tree and single-step reaction template prediction in Example 1. Therefore, based on the method introduced in Example 1, those skilled in the art can understand the specific structure of the system, which is not repeated here. All systems used in the method of Example 1 of the present disclosure belong to the scope of protection of the present disclosure.

It should be understood that the above description of the preferred embodiments is relatively detailed, and therefore should not be considered as limiting the scope of the patent protection of the present disclosure. Under the inspiration of the present disclosure, those of ordinary skill in the art can also make substitutions or modifications without departing from the protection scope of the present disclosure, which all fall within the protection scope of the present disclosure. The scope of the claimed protection of the present disclosure shall be subject to the appended claims.

What is claimed is:

1. A bioretrosynthetic method based on an AND-OR tree and single-step reaction template prediction, comprising the following steps:
S1: selecting an OR node from a pre-constructed AND-OR tree, and using a molecule corresponding to the OR node as a product molecule to be predicted; wherein the pre-constructed AND-OR tree comprises two types of nodes: AND node and OR node; the AND node represents a reaction template, and the OR node represents a molecule;
S2: predicting k templates in a preset template set that are most likely to synthesize the product molecule using a pre-constructed single-step reaction template prediction model, forming a template set Top-k, and assigning a weight value ranged between 0 and 1 to each template; wherein the preset template set is constructed based on a metabolic reaction structure in a known metabolic reaction data set;
S3: expanding the pre-constructed AND-OR tree, specifically comprising: adding each template in the Top-k as a new AND node to the AND-OR tree to obtain newly-added k AND nodes, and using the OR node selected in step S1 as a parent node of the newly-added k AND nodes; adding each reaction substrate molecule corresponding to each newly-added AND node to the AND-OR tree as an OR node to obtain newly-added OR nodes, with the newly-added AND node as a parent node of the newly-added OR nodes; and
S4: determining whether there is an AND node in step S3, wherein a substrate molecule corresponding to a child node of the AND node belongs to a known metabolite set: Sink-Compounds set; if there is an AND node, finding a biosynthetic pathway, stopping an iterative retrosynthetic process, and generating the biosynthetic pathway; and if there is no AND node, determining whether the maximum number of iterations has been reached; if the maximum number of iterations is reached, stopping the iterative retrosynthetic process; and if the maximum number of iterations is not reached, repeating steps S1 to S4 until a biosynthetic pathway is found or the maximum number of iterations is reached.

2. The bioretrosynthetic method according to claim 1, wherein in step S1: in the AND-OR tree, a root node and a leaf node each are an OR node, and an intermediate node is an AND node or an OR node; a child node of each AND node is an OR node, representing all substrate molecules in a reaction template; a child node of each non-leaf OR node in the AND-OR tree is an AND node; each AND node represents a reaction template capable of producing a molecule corresponding to a parent node of the AND node, and the root node of the AND-OR tree is a target molecule node for quasi-prediction of a biosynthetic pathway; and an initially-constructed AND-OR tree comprises only one root node, corresponding to the target molecule of a biosynthetic pathway to be predicted.

3. The bioretrosynthetic method according to claim 1, wherein in step S1, each node of the AND-OR tree has a weight value; a weight value of the AND node is a weight value of a corresponding reaction template, indicating a prediction probability of the corresponding reaction template; and OR nodes other than the root node each have a weight value of the parent node except for the OR nodes, and the root node has a weight value of 1.

4. The bioretrosynthetic method according to claim 3, wherein in step S1, the selected OR node is a leaf node in the AND-OR tree that does not belong to the known Sink-Compounds set and has a maximum weight value; and if there are a plurality of the leaf nodes with the maximum weight value, one of the leaf nodes is selected randomly as the OR node.

5. The bioretrosynthetic method according to claim 1, wherein the pre-constructed single-step reaction template prediction model is a multi-classification model based on machine learning; and step S2 specifically comprises:
S2.1: predicting a probability that all reaction templates in the preset template set are capable of producing an input product molecule using the constructed multi-classification model; and
S2.2: selecting top k reaction templates with the highest probabilities to form the template set Top-k, and setting a weight value of each reaction template in the template set Top-k as a corresponding probability value.

6. The bioretrosynthetic method according to claim 1, wherein in step S3, each reaction substrate molecule of the newly-added AND nodes is obtained by calling a function in an open source library RDChiral; and in the function, an input parameter is a SMILES sequence of the reaction template and the product molecule, and an output is a list of the corresponding substrate molecules.

7. The bioretrosynthetic method according to claim 1, wherein in step S4, a process of generating the biosynthetic pathway comprises the following steps:
(1) checking whether each leaf node in the AND-OR tree is in the Sink-Compounds set; marking the leaf node in the Sink-Compounds as “true”, and marking the leaf node not in the Sink-Compounds as “false”;
(2) for a non-leaf AND node in the AND-OR tree, marking the non-leaf AND node as “true” if and only if each child node of the non-leaf AND node is marked as “true”, otherwise marking the non-leaf AND node as “false”; and for a non-leaf OR node in the AND-OR tree, marking the non-leaf OR node as “true” if and only if the non-leaf OR node comprises at least one child node marked as “true”, otherwise marking the non-leaf OR node as “false”; and
(3) if the root node is marked as “false”, it indicates that no synthetic pathway has been found, outputting “No Solution”; otherwise, deleting all nodes marked as “false” in the AND-OR tree, and remaining a subtree representing a synthetic pathway of a target molecule.

8. A bioretrosynthetic system based on an AND-OR tree and single-step reaction template prediction, comprising:
a retrosynthesis planning module used for selecting an OR node from a pre-constructed AND-OR tree, and using a molecule corresponding to the OR node as a product molecule to be predicted; wherein the pre-constructed AND-OR tree comprises two types of nodes, an AND node and the OR node; the AND node represents a reaction template, and the OR node represents a molecule;
a reaction template prediction module used for predicting k templates in a preset template set that are most likely to synthesize the product molecule using a pre-constructed single-step reaction template prediction model, forming a template set Top-k, and assigning a weight value ranged between 0 and 1 to each template; wherein the preset template set is constructed based on a metabolic reaction structure in a known metabolic reaction data set;
an AND-OR tree extension module used for expanding the pre-constructed AND-OR tree, specifically comprising: adding each template in the Top-k as a new AND node to the AND-OR tree to obtain newly-added k AND nodes, and using the OR node selected in the retrosynthesis planning module as a parent node of the newly-added k AND nodes; adding each reaction substrate molecule corresponding to each newly-added AND node to the AND-OR tree as an OR node to obtain newly-added OR nodes, with the newly-added AND node as a parent node of the newly-added OR nodes; and
a biosynthetic pathway generation module used for determining whether there is an AND node in the AND-OR tree obtained by the AND-OR tree extension module, wherein a substrate molecule corresponding to a child node of the AND node belongs to a known metabolite set: Sink-Compounds set; if there is an AND node, finding a biosynthetic pathway, stopping an iterative retrosynthetic process, and generating the biosynthetic pathway; if there is no AND node, determining whether the maximum number of iterations has been reached; if the maximum number of iterations is reached, stopping the iterative retrosynthetic process; if the maximum number of iterations is not reached, repeating steps of the retrosynthesis planning module to the biosynthetic pathway generation module until a biosynthetic pathway is found or the maximum number of iterations is reached.

9. The bioretrosynthetic system according to claim 8, further comprising: a biosynthetic pathway visualization module for visually displaying an obtained biosynthetic pathway of a target molecule.
